(19) United States
(12) Patent Application Publication
(10) Pub. No.: US 2025/0049295 A1
(43) Pub. Date: Feb. 13, 2025

(54) METHODS OF LIMITING POWER TO A PORTABLE/HAND-HELD DEVICE

(71) Applicant: CANON U.S.A., INC., MELVILLE, NY (US)

(72) Inventor: Daisuke YAMADA, Cambridge, MA (US)

(21) Appl. No.: 18/718,285

(22) PCT Filed: Dec. 7, 2022
(86) PCT No.: PCT/US22/81120

§ 371 Date: Jun. 10, 2024

Related U.S. Application Data
(60) Provisional application No. 63/287,702, filed on Dec. 9, 2021.

(51) Int. Cl. A61B 1/00

(52) U.S. Cl. CPC A61B 1/00036; A61B 1/0016

(57) ABSTRACT

An articulated medical device having a hollow core, capable of large degrees of maneuverability through small cavities to reach a target with minimal invasiveness, wherein the medical device is capable of manual and robotic manipulation.

### CROSS REFERENCE TO RELATED PATENT APPLICATIONS

This application claims priority from U.S. Provisional Patent Application No. 63/287,702, filed on Dec. 9, 2021, in the United States Patent and Trademark Office, the disclosure of which is incorporated by reference herein, in its entirety.

### FIELD OF THE DISCLOSURE

The present disclosure relates generally to apparatus and methods for medical application. More particularly, the subject disclosure is directed to medical devices and methods for limiting the power to a medical device to diminish the risk of harm caused by the patient interface unit (“PIU”) and device, while allowing for uninterrupted use of the medical device.

### BACKGROUND OF THE DISCLOSURE

In a typical catheter/endoscope based imaging system, imaging of the information obtained from the endoscope is accomplished by utilizing a patient interface unit (PIU) connected between the catheter and the system console. The PIU comprises of a fiber optic rotary joint, a rotational motor, a translational motor and drivers for each of the two motors.

In order to acquire cross-sectional images of various internal cavities such as vessels, the esophagus, and nasal cavities, the optical probe is rotated with a fiber optic rotary joint (FORJ) with the rotational motor. While the optical probe is being rotated, the optical probe is simultaneously translated longitudinally during the rotation with the translational motor, so that helical scanning pattern images are obtained. This translation is most commonly performed by pulling the tip of the probe back through the internal cavity and therefore referred to as a “pullback”.

The PIU is placed in the subject's vicinity, and accordingly, requires compliance with various standards for safety. One such requirement is complying with flammable safety requirements under single fault conditions to minimize risk of the harm to the subject, physicians, staff and the environment.

One such standard is IEC 60601-1, which addresses medical electrical equipment, wherein part 1 is for General requirements for basic safety and essential performance. As this standard has been adopted by countries the world over as the benchmark for the import of medical devices. The standard is highly relevant and worth maximizing.

More specifically, the IEC 60601-1 standard states “The single fault conditions in . . . shall not be applied to parts and components where: the construction of the supply circuit limits the power dissipation in single fault condition to less than 15 W or the energy dissipation to less than 900 Joules. Compliance is checked by drawing 15 W from the supply circuit for 1 minute. If after 1 minute the supply circuit cannot supply 15 W the circuit shall be considered to limit power dissipation to less than 15 W. The related design documentation is also reviewed.” (Note: 900 joules is the area under the curve of 15 W for 60 seconds).

Most imaging systems incorporate a stand-by mode and an imaging mode. In the standby mode, the system is on stand-by and waiting for the end user's interaction, whereby the motors in the PIU are not activated. In stand-by mode the power consumption of the PIU is less than 15 W or 900 J. On the other hand, in the imaging mode, the system is displaying and/or recording images, and the motor(s) in the PIU are active. Thus, power consumptions of the PIU exceed 15 W and/or 900 J while in the imaging mode. However, as the imaging mode is limited to less than 60 seconds, the current state of the art limits the supplied power circuit to the PIU, using a current threshold limiter such as a fuse or monitoring current with a trip meter when exceeding predetermined current limit. However, the current state of the art is deficient in that the supplied power is interrupted while the imaging mode is in progress, thus rendering an incomplete pull-back image of the target cavity.

In order to comply with the IEC 60601-1 standards while optimizing power efficiency, and gaining a full view of the specimen throughout the pullback, the subject innovation has been developed.

### SUMMARY

Thus, to address such exemplary needs in the industry, the present disclosure provides a medical imaging device and associated methods and systems for employing the device, wherein the medical imaging system comprises a patient interface; a console in communication with the patient interface unit and including a power supply module for supplying power to the patient interface, an imaging engine, and a display, wherein the patient interface comprises: at least one of a rotational motor and/or a translational motor; and at least one driver for operating the rotational motor and/or translation motor, wherein the power supply module is configured with a first power threshold which is restricted to 15 or more watts of power for under sixty seconds.

In various embodiments, the system may further comprise a catheter in communication with the patient interface unit.

In yet additional embodiments, the patient interface within the system further comprises a fiber optic rotary joint.

It is also contemplated that the power supply module may be configured with a second power threshold which is greater than the first power threshold, and wherein the power supply module is restricted once the second power threshold is exceeded for a specific duration of time. In further of that, the specific duration of time may be defined as more than 1 minute.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present invention will become apparent from the following detailed description when taken in conjunction with the accompanying figures showing illustrative embodiments of the present invention.

FIG. 1 illustrates an OCT and fluorescence system, according to one or more embodiment of the subject apparatus, method or system.

FIG. 2 provides a schematic signal flow for an OCT and fluorescence system, according to one or more embodiment of the subject apparatus, method or system.

FIG. 3 depicts a block diagram of the supplied power module, according to one or more embodiment of the subject apparatus, method or system.

FIG. 4 provides a schematic of the supplied power module, according to one or more embodiment of the subject apparatus, method or system.

FIG. 5 also provide a schematic of the supplied power module in association with FIG. 4, according to one or more embodiment of the subject apparatus, method or system.

FIG. 6 depict the patient interface unit, according to one or more embodiment of the subject apparatus, method or system.

FIG. 7 is a side cut-away image of a catheter, according to one or more embodiment of the subject apparatus, method or system.

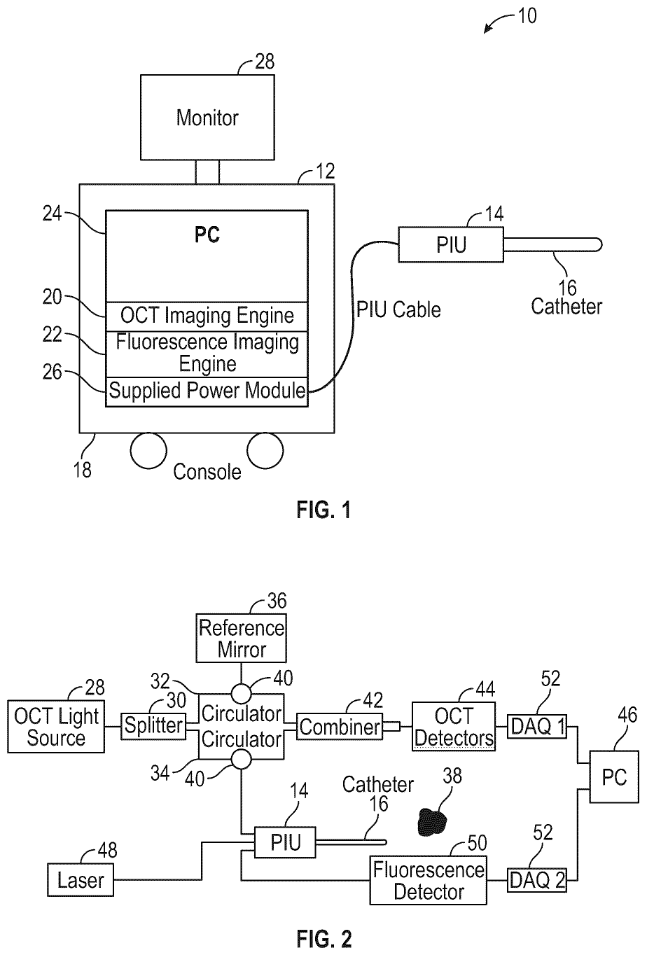

Throughout the Figures, the same reference numerals and characters, unless otherwise stated, are used to denote like features, elements, components or portions of the illustrated embodiments. In addition, reference numeral(s) including by the designation “′” (e.g. 12′ or 24′) signify secondary elements and/or references of the same nature and/or kind. Moreover, while the subject disclosure will now be described in detail with reference to the Figures, it is done so in connection with the illustrative embodiments. It is intended that changes and modifications can be made to the described embodiments without departing from the true scope and spirit of the subject disclosure as defined by the appended paragraphs.

### DETAILED DESCRIPTION OF THE DISCLOSURE

Exemplary embodiments are described below with reference to the drawings. The following description and drawings are illustrative of the disclosure and are not to be construed as limiting the disclosure.

In referring to the description, specific details are set forth in order to provide a thorough understanding of the examples disclosed. In other instances, well-known methods, procedures, components and materials have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description and/or illustration to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. As used herein, the singular forms “a”, “an”, and “the”, are intended to include the plural forms as well, unless the context clearly indicates otherwise. It should be further understood that the terms “includes” and/or “including”, when used in the present specification, specify the presence of stated features, integers, steps, operations, elements, and/or components, but do not preclude the presence or addition of one or more other features, integers, steps, operations, elements, components, and/or groups thereof not explicitly stated. The term “position” or “positioning” should be understood as including both spatial position and angular orientation.

FIG. 1 illustrates an exemplary OCT and fluorescence multi-modality imaging system 10 that comprises a console 12 with a monitor 28, a PIU 14 and a catheter 16.

The console 12 may be assembled upon a mobile cart 18 that houses the OCT imaging engine 20, the fluorescence imaging engine 22, the host computer 24 and the supplied power module 26.

The OCT imaging engine 20 includes an OCT light source 28 with a wavelength of around 1.3 um, delivered from a light source and split into a reference arm 32 and a sample arm 34 with a splitter 30. The split light from the reference arm 32 provided a reference beam, which is reflected from a reference mirror 36 in the reference arm 32. While a sample beam provided by the sample arm 34 is reflected and/or scattered from a sample through a PIU 14 and a catheter 16 in the sample arm 34. Fibers of the PIU 14 and catheter 16 are made of a DCF (double clad fiber). The OCT light illuminates the sample 38 through the core of DCF, and scattered light from the sample 38 are collected and delivered back to the circulator of an OCT interferometer 40 via the PIU, and combined with the reference beam at the combiner 42 to generate interference patterns.

The output of the interferometer is detected with the OCT detectors 44 such as photodiodes or multi-array cameras. Then signals are transferred to a computer 46 to perform signal processing to generate OCT images. The interference patterns are generated only when the path length of the sample arm matches that of the reference arm to within the coherence length of the light source.

The fluorescence imaging engine 22 includes a laser 48 having an approximate wavelength of 0.635 um, which originates from a fluorescence light source and is delivered to the sample 38 through the PIU 14 and the catheter 16. The patient interface unit (PIU, explained herein) includes a free space beam combiner so that the excitation light couples into the common DCF with OCT.

The laser 48 of the fluorescence imaging engine 22 illuminates the sample 38 from a distal end of the optical probe in the catheter 16. The sample 38 emits auto-fluorescence within the broadband wavelengths of 0.65-0.90 um. The auto-fluorescence and the Raman are delivered to a fluorescence detector 50, such as photo-multi plier tube (PMT), via the PIU 14. Then, the analog electrical signal at the fluorescence detector 50 is acquired by a data acquisition board (DAQ 2) 52.

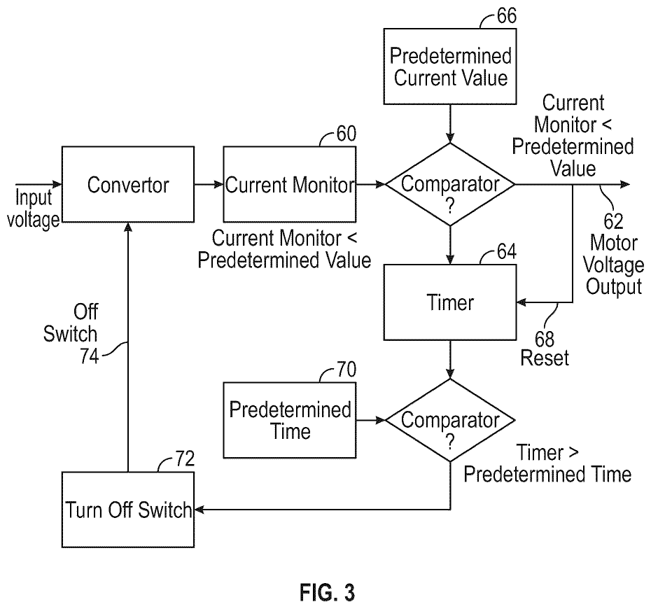

FIG. 3 details the supplied power module 26 which supplies two voltage powers (logic power, motor power) to the PIU 14 via the PIU cable. The two voltage powers are the logic voltage output for the logic circuits and the motor voltage output for driving motors, respectively. A current monitor 60 on the motor voltage output 62 is placed to monitor the supplied power, which is calculated by voltage times the current.

The current monitor 60 on the motor voltage output starts a timer 64 if the current exceed a predetermined threshold 66. If the current drops below the threshold, the timer will reset 68. If the timer 68 reaches a predetermined time 70 the motor voltage will be shut off 72 using a power switch 74, shown in FIG. 3.

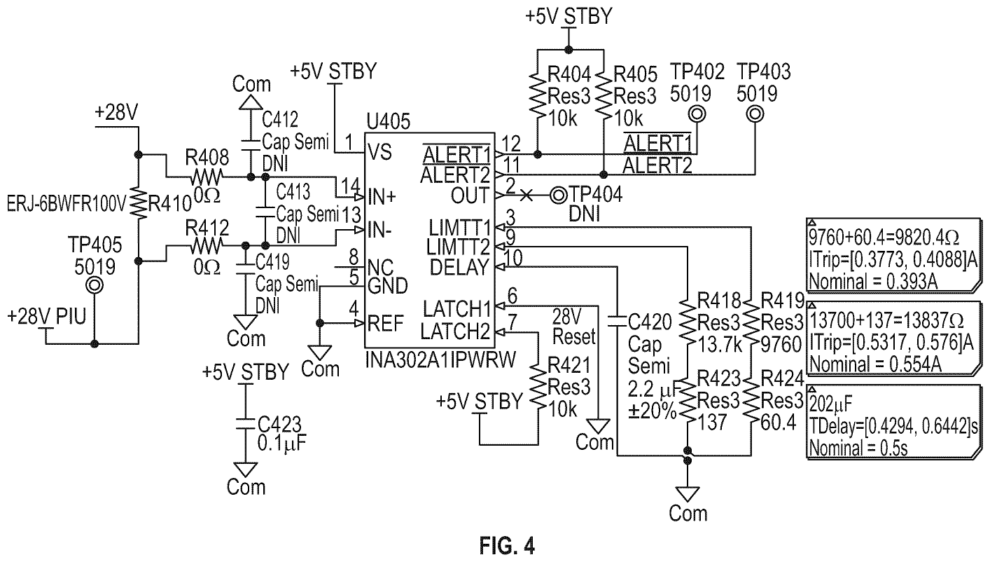

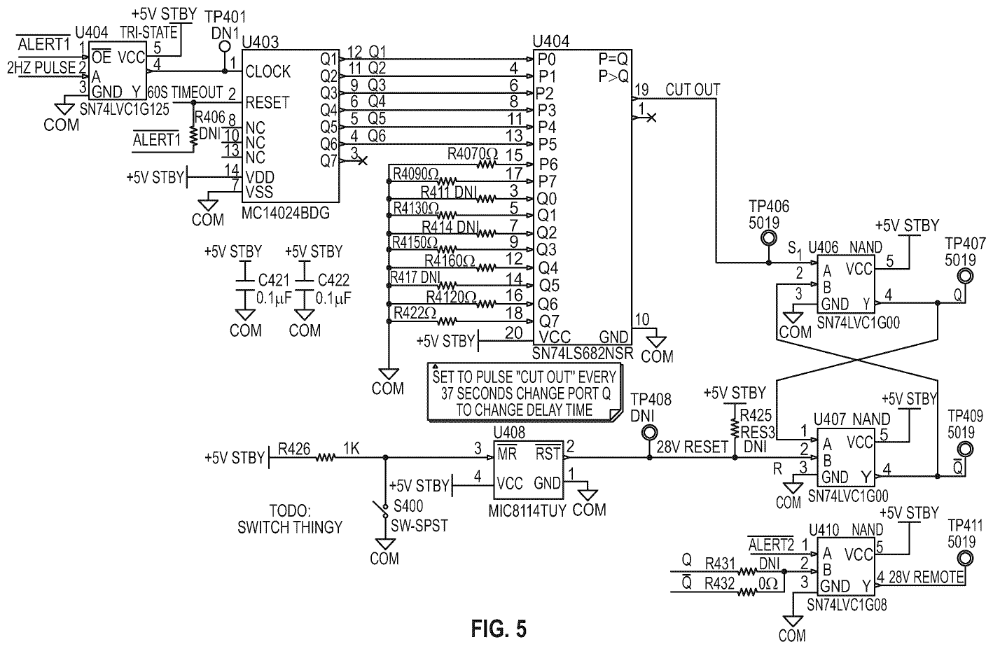

FIG. 4 shows implementation of the above-mentioned supplied power module, wherein the high-side current monitor is designed around an INA302 A integrated circuit (“IC”) which monitors the motor voltage current via the voltage drop across a 0.1 ohm resistor, and translates the voltage drop down to ground level. The INA302 A has two current limits. Limit-1 uses an on-board timer while Limit-2 trips without time delay. The limits and timer are set for:
Limit-1 generates the Alert-1 signal, which is timed and latched. This time is set to 37 seconds.
Limit-2 generates the Alert-2 signal and is latched by the INA302A.

A current monitor on the motor voltage output of 28V starts a timer if the 28V current exceeds 0.5 A (15 W); if the current drops below 0.5 A the timer will reset. If the timer reaches 37 seconds the 28V converter will be turned off and latched off. The timer limits the delivered energy −28V times 0.5 A (14 W) for 37 seconds is 518 joules, which is below the limit of 900 J. When tested with a maximum power of 15 W the output will time-out before 60 seconds. The over-current condition is latched and requires that the system be powered off to reset.

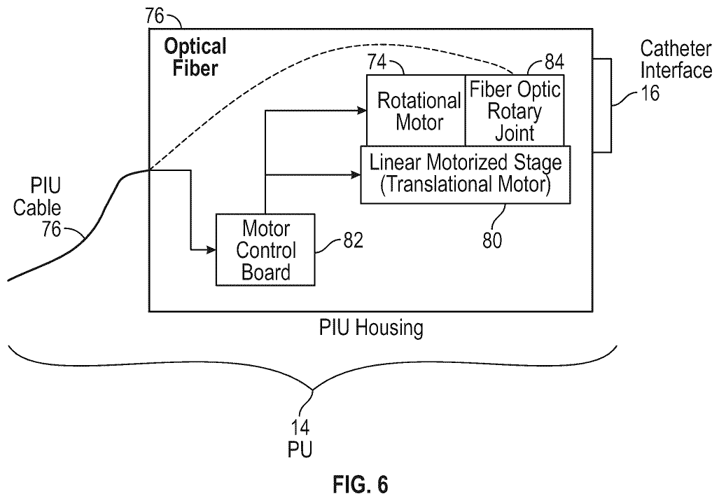

With reference to FIG. 6, the PIU 14 is an interface between the console 12 and the catheter 16. The PIU 14 provides the means to spin and linearly translate the catheter's 16 optical probe within the catheter's stationary outer sheath. The console 12 and PIU 14 are connected by the PIU cable 76, which houses electrical power, a communication cable and optical fibers 78. The PIU 14 comprises a rotational motor 74, translational motor 80, fiber optic rotary joint 84 and the motor control board(s) 82. The motors are activated by the control board during imaging and recording. The power of the motors 74 and 80 and the control board 82 are supplied by the supplied power module 26 in the console 12 via the PIU cable 76.

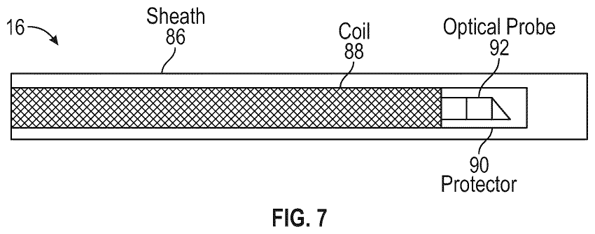

The catheter 16 can been seen in FIG. 7 and comprises a sheath 86, a coil 88, a protector 90 and an optical probe 92. The catheter 16 is connected to the PIU 14, shown in FIG. 6. The optical probe 92 comprises an optical fiber connector 78, an optical and distal lens. The optical fiber connector 78 is used to engage with the PIU 14, and the optical fiber is to deliver light to the distal lens. The distal lens focuses the optical beam to illuminate light upon the sample 38, as well as to collect light reflected from the sample 38 efficiently.

The coil 88 delivers the torque from the proximal end of the catheter 16 to the distal end by a rotational motor 74 in the PIU 14. There is a mirror at the distal end of the catheter 16 so that the light beam is deflected outward. The coil 88 is fixed with the optical probe 92 so that a distal tip of the optical probe 92 also spins to allow for omnidirectional viewing of the inner surface of hollow organs, such as vessels. The optical probe 92 comprises a fiber connector at the proximal end, double clad fiber and a lens at the distal end. The fiber connector is connected with the PIU 14.

Measurement workflow is detailed in this section. Once the imaging system 10 is powered up and the application software is launched, the imaging system 10 is idle and waits for a catheter 16 connection. During the idle state, the motors 74 and 80 of the PIU 14 are not activated so that the monitored current of the motor voltage output in the supplied power module 26 will not exceed the predetermined threshold current 66.

When the user connects the catheter 16 to the PIU 16, the system 10 automatically engages the catheter 16 optically and mechanically by translating linear motorized stage 80 while the rotational motor 74 is not spinning. The engagement process will not take more than 37 seconds. The current of the motor voltage output will not exceed the predetermined threshold 66 because of the stationary state of the rotational motor 74. Even if the motor voltage exceeds the predetermined threshold 66, the engagement process time is shorter than the predetermined time 70 so that the timer 64 of the over current chip in the supplied power module 26 will be reset 68.

Once the catheter 16 is engaged, the system 10 is in standby mode, wherein both the rotational motor 74 and the translational motor 80 are stationary, and the current of the motor voltage output in the supplied power module 26 is less than the predetermined threshold 66.

In the live-view image (real-time image) state, the catheter 16 is spinning without the linear motion wherein the translational motor 80 can be enacted after the user's response. The current of the motor voltage output in this state will not exceed the predetermined threshold 66 because of the stationary state of the rotational 74 motor.

Finally, in the pullback imaging state, the catheter 16 records images of the cavities. In this imaging state, both the rotational motor 74 and the translational motor 80 are activated, so that the current of the motor voltage output will exceed the predetermined threshold 66. However, the pullback imaging time is less than the predetermined time of 37 seconds, so that the timer 64 of the over current chip in the supplied power module 26 will be reset 68.

The relationship between time (T), voltage (V) and current (I) used herein is represented by: V*I*T<900 J, where V is supplied voltage from the power supply.

In keeping this relationship in mind, if time is going to be less than or equal to 60 seconds: the first threshold current limit is less than or equal to A*900/V/60=15*A/V, where A is 0.5 to 1.0; and the second threshold current limit is less than or equal to 900/V/60=15/V.

If time is between 30 and 60 seconds: the first threshold current limit is less than or equal to A*900/V/T, where A is 0.5 to 1.0; and the second threshold current limit is less than or equal to 900/V/T. Thus satisfying the parameters set by the IEC.

In summation, the system 10, and more specifically the PIU 14, can minimize the risk of flammability by limiting the current of the motor voltage in the supplied power module 26 within the predetermined power and time. The subject innovation allows the system 10 to meet the flammability requirement of IEC 60601-1 with the use of two power (current, voltage) and time thresholds without hindering the systems performance.

While the present disclosure has been described with reference to exemplary embodiments, it is to be understood that the disclosure is not limited to the sample exemplary embodiments provided herein. The scope of the following claims is to be accorded the broadest interpretation so as to encompass all such modifications and equivalent structures and functions.

1. A medical imaging system comprising:
a patient interface;
a console in communication with the patient interface and including a power supply module for supplying power to the patient interface, an imaging engine, and a display,
wherein the patient interface comprises:
at least one of a rotational motor and/or a translational motor; and
at least one driver for operating the rotational motor and/or translation motor,
wherein the power supply module is configured with a first power threshold which is restricted to fifteen or more watts of power for under sixty seconds.

2. The system of claim 1, further comprising a catheter in communication with the patient interface unit.

3. The system of claim 1, wherein the patient interface further comprises a fiber optic rotary joint.

4. The system of claim 1, wherein the power supply module is configured with a second power threshold which is greater than the first power threshold, and wherein the power supply module is restricted once the second power threshold is exceeded for a specific duration of time.

5. The system of claim 4, wherein the specific duration of time is more than one minute.

6. A medical imaging apparatus comprising:
a patient interface;
a console in communication with the patient interface and including a power supply module for supplying power to the patient interface, an imaging engine, and a display,
wherein the patient interface comprises:
at least one of a rotational motor and/or a translational motor; and
at least one driver for operating the rotational motor and/or translation motor,
wherein the power supply module is configured with a first power threshold.

7. The apparatus of claim 6, wherein the first power threshold is restricted to less than sixteen watts of power.

8. The apparatus of claim 6, wherein the first power threshold is utilized after a duration of time for supplying power to the imaging engine is more than one minute.

9. The apparatus of claim 6, further comprising a catheter in communication with the patient interface unit.

10. The apparatus of claim 6, wherein the patient interface further comprises a fiber optic rotary joint.

11. The apparatus of claim 6, wherein the power supply module is configured with a second power threshold which is greater than the first power threshold, and wherein the power supply module is restricted once the second power threshold is exceeded for a specific duration of time.

12. The apparatus of claim 11, wherein the specific duration of time is more than 1 minute.
